(19) United States
(12) Patent Application Publication
(10) Pub. No.: US 2014/0257201 A1
(43) Pub. Date: Sep. 11, 2014

(54) SAFETY SYSTEM FOR A SYRINGE

(71) Applicants: Joerg Geiger, Gais (CH); C. Garyen Denning, Lexington, KY (US); David Schmidt, Nicholasville, KY (US); Theodore J. Mosler, Raleigh, NC (US); Andrew J. Corson, Apex, NC (US); Edward P. Browka, Chapel Hill, NC (US)

(72) Inventors: Joerg Geiger, Gais (CH); C. Garyen Denning, Lexington, KY (US); David Schmidt, Nicholasville, KY (US); Theodore J. Mosler, Raleigh, NC (US); Andrew J. Corson, Apex, NC (US); Edward P. Browka, Chapel Hill, NC (US)

(21) Appl. No.: 14/204,959

(22) Filed: Mar. 11, 2014

Related U.S. Application Data
(60) Provisional application No. 61/776,414, filed on Mar. 11, 2013.

(51) Int. Cl. A61M 5/32

(52) U.S. Cl. CPC A61M 5/3204
USPC 604/263; 29/428

(57) ABSTRACT

A safety system including a needle retaining device that can include a proximal end, a distal end, and a needle fixedly coupled to the distal end of the needle retaining device. The safety system can include a needle guard assembly, where the needle guard assembly can be configured for attachment to the distal end of the needle retaining device. The needle guard assembly can include a first cylinder, where the first cylinder can be attached to the distal end of the needle retaining device, and a second cylinder, where the second cylinder can be selectively movable relative to the first cylinder from a first position, to a second position wherein the needle is fully exposed, to a third position wherein the needle is fully covered.

### REFERENCE TO RELATED APPLICATION

The present application claims the priority benefit of U.S. provisional patent application Ser. No. 61/776,414, filed Mar. 11, 2013, which is hereby incorporated by reference in its entirety.

### TECHNICAL FIELD

Embodiments of the technology relate, in general, to safety syringes, and in particular to passive safety systems that can be attached to a standard syringe to prevent accidental needlesticks.

### BRIEF DESCRIPTION OF THE DRAWINGS

The present disclosure will be more readily understood from a detailed description of some example embodiments taken in conjunction with the following figures:

FIG. 1 depicts a side view of an example safety system shown attached to a syringe according to one embodiment.

FIG. 2 depicts a side cross-sectional view of the safety system shown in FIG. 1, taken along A-A.

FIG. 3 depicts a perspective view of the safety system shown in FIG. 1 at a first angle.

FIG. 4 depicts a perspective view of the safety system shown in FIG. 1 taken at a second angle.

FIG. 5 depicts a partial side view of the safety system shown in FIG. 1, where the safety system is shown with a portion of the first cylinder cut away in an initial position according to one embodiment.

FIG. 6 depicts a partial side view of the safety system shown in FIG. 1, where the safety system is shown with a portion of the first cylinder cut away in an armed position according to one embodiment.

FIG. 7 depicts a partial side view of the safety system shown in FIG. 1, where the safety system is shown with a portion of the first cylinder cut away in an extended position according to one embodiment.

FIG. 8 depicts a perspective view of the safety system shown in FIG. 5, where the safety system is shown with a portion of the first cylinder cut away in an initial position according to one embodiment.

FIG. 9 depicts a perspective view of the safety system shown in FIG. 6, where the safety system is shown with a portion of the first cylinder cut away in an armed position according to one embodiment.

FIG. 10 depicts a perspective view of the safety system shown in FIG. 7, where the safety system is shown with a portion of the first cylinder cut away in an extended position according to one embodiment.

### SUMMARY

A safety system including a needle retaining device that can include a proximal end, a distal end, and a needle fixedly coupled to the distal end of the needle retaining device. The safety system can include a needle guard assembly, where the needle guard assembly can be configured for attachment to the distal end of the needle retaining device. The needle guard assembly can include a first cylinder, where the first cylinder can be attached to the distal end of the needle retaining device, and a second cylinder, where the second cylinder can be selectively movable relative to the first cylinder from a first position, to a second position wherein the needle is fully exposed, to a third position wherein the needle is fully covered.

A method for assembling a safety system can include providing a needle retaining device that can have a proximal end, a distal end, and a needle fixedly coupled to the distal end of the needle retaining device. The method can include providing a needle guard assembly, where the needle guard assembly can be configured for attachment to the distal end of the needle retaining device. The needle guard assembly can include a first cylinder, where the first cylinder can be attached to the distal end of the needle retaining device, and a second cylinder, where the second cylinder can be selectively movable relative to the first cylinder from a first position, to a second position wherein the needle is fully exposed, to a third position wherein the needle is fully covered. The method can include coupling the needle guard assembly and the needle retaining device.

### DETAILED DESCRIPTION

Various non-limiting embodiments of the present disclosure will now be described to provide an overall understanding of the principles of the structure, function, and use of the safety systems and processes disclosed herein. One or more examples of these non-limiting embodiments are illustrated in the accompanying drawings. Those of ordinary skill in the art will understand that systems and methods specifically described herein and illustrated in the accompanying drawings are non-limiting embodiments. The features illustrated or described in connection with one non-limiting embodiment may be combined with the features of other non-limiting embodiments. Such modifications and variations are intended to be included within the scope of the present disclosure.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment, or “in an embodiment” in places throughout the specification are not necessarily all referring to the same embodiment. Furthermore, the particular features, structures or characteristics may be combined in any suitable manner in one or more embodiments.

The examples discussed herein are examples only and are provided to assist in the explanation of the apparatuses, devices, systems and methods described herein. None of the features or components shown in the drawings or discussed below should be taken as mandatory for any specific implementation of any of these the apparatuses, devices, systems or methods unless specifically designated as mandatory. For ease of reading and clarity, certain components, modules, or methods may be described solely in connection with a specific figure. Any failure to specifically describe a combination or sub-combination of components should not be understood as an indication that any combination or sub-combination is not possible. Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

Example embodiments described herein can be used to prevent accidental needlesticks. Example embodiments can include a safety system that can be attached to a standard off the shelf syringe or pre-filled cartridge. Embodiments can be Integrated into a standard prefilled syringe by, for example, a glass company. Embodiments can include automatic, passive needlestick safety protection that can comply with US and EU guidelines for needlestick safety prevention. Embodiments fo the safety system can use current stability packaging materials of a validated PFS. Embodiments can provide a needlestick safety solution that may be implemented with lower total cost than, for example, bolt-on safety solutions.

Example embodiments described herein can be used with a standard needle, a standard glass hub, a standard cone, a standard needle shield, or a standard fixture to the glass hub of a syringe or pre-filled cartridge. Embodiments can include a one-step assembly of the safety system by a manufacture, where, for example, a manufacturer can pre-assemble the safety system. Embodiments can fit in standard three inch or four inch tubs and can fit in 10×16 or 10×10 tubs, for example.

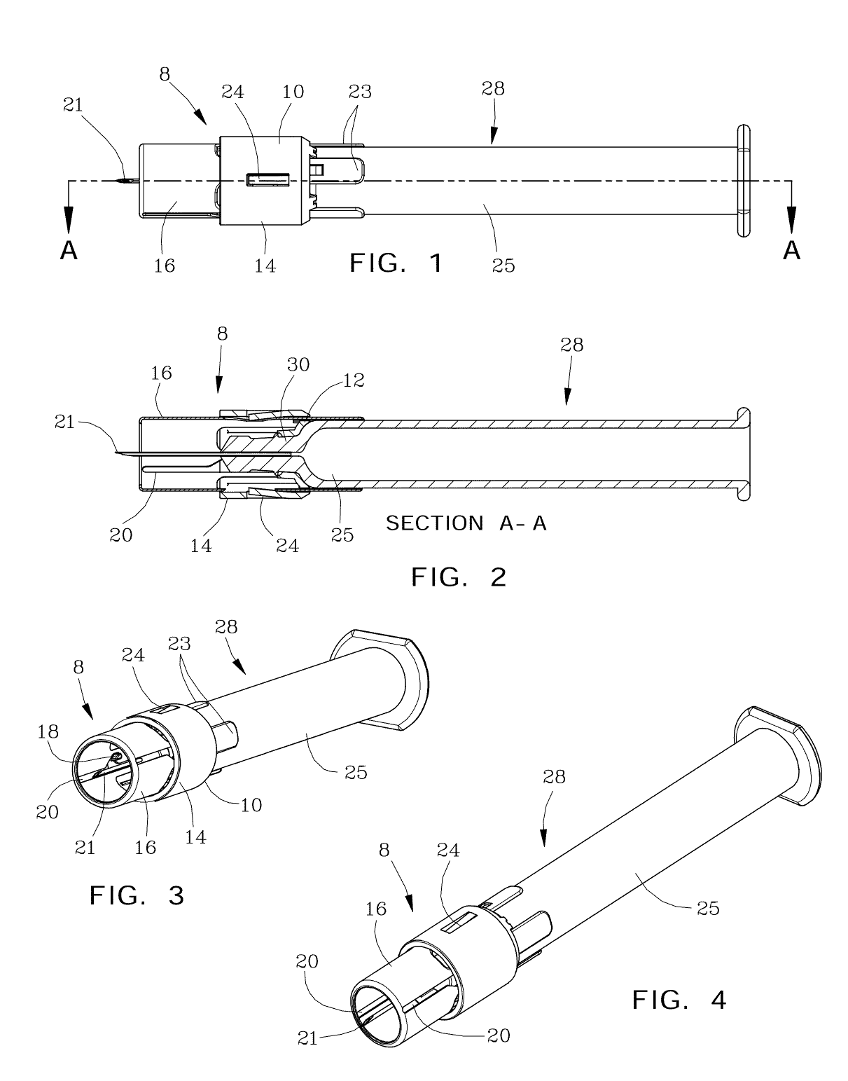

Referring now to FIGS. 1-10, an automatic non-reusable needle guard 8 can include a first cylinder 10 that can be attached to a needle retaining device 28, such as a standard syringe or pre-filled cartridge that can retain a needle 21. The first cylinder 10 can have an inner portion 12 and an outer portion 14. In an example embodiment, the inner portion 12 can be coupled with the neck 30 of the standard needle retaining device 28 with a snap fit, friction fit, or any other suitable coupling. It will be appreciated that needle guard 8 can have any suitable shape, size, or configuration.

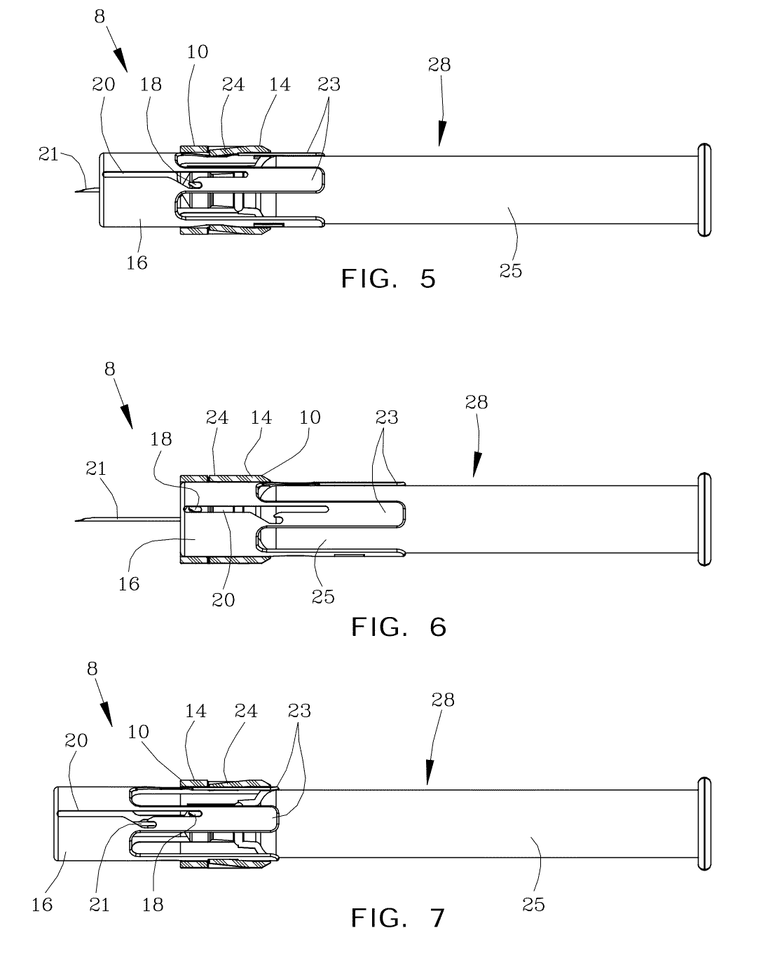

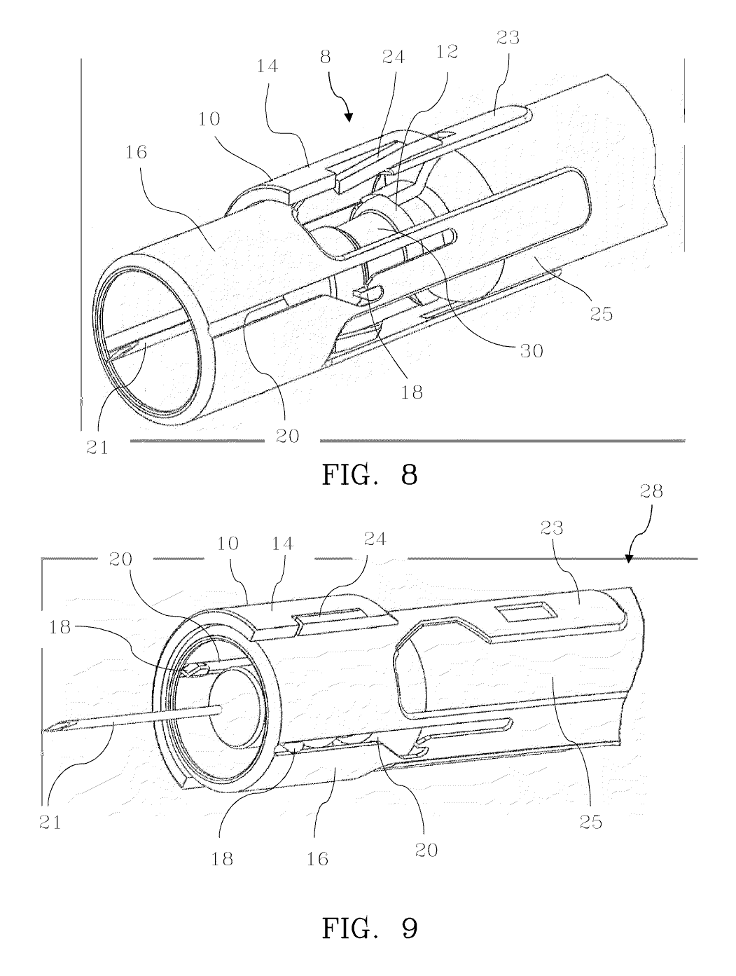

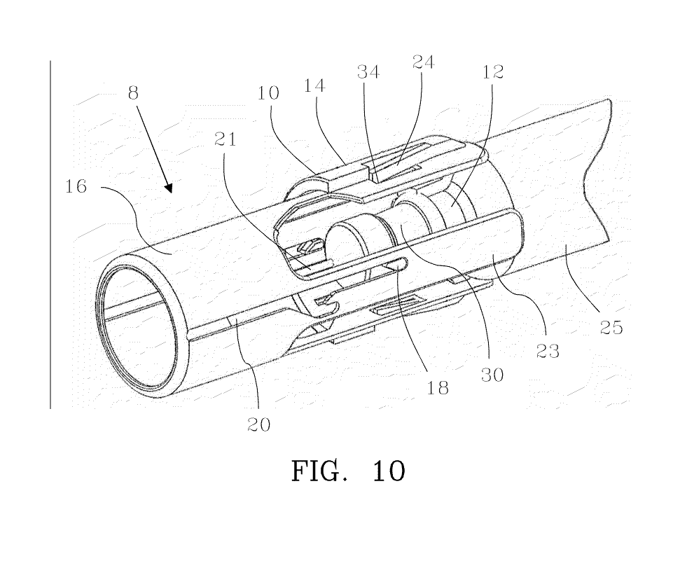

The automatic non-reusable needle guard 8 can include a second cylinder 16 that can be operable with the first cylinder 10 for movement of the second cylinder 16 from an initial retractable position (FIGS. 5 and 8) to a released or armed position (FIGS. 6 and 10) and further to an extended irreversible position (FIGS. 7 and 10). The second cylinder 16 can include a plurality of flanges 23 that can extend axially along a portion of the needle retaining device 28. The flanges may help stabilize the needle guard 8 relative to the needle retaining device 28. In an alternate embodiment, the needle guard 8 may not have any flanges where, for example, no portion of the needle guard 8 can project proximally over the body 25 of the needle retaining device 28. Such an alternate configuration may facilitate visualization of the contents within the needle retaining device 28.

The mechanism that can provide movement of the second cylinder 16 relative to the first cylinder 10 can incorporate a first track 20, as shown in more detail in FIGS. 5-10, or a plurality of tracks, where the one or a plurality of tracks can have any suitable shape, such as a J-shape or a generally linear shape having a lateral track projection. The outer portion 14 of the first cylinder 10 can incorporate a first protruding member 18 (FIGS. 3 and 5-10) which can be formed on the outer portion 14 of the first cylinder 10 and can be positioned in the first track 20 such that the first track can guide the second cylinder from an initial usable position to a released position and further to an extended irreversible position. A spring (not shown) can be disposed between the second cylinder 16 and the inner portion 12 of the first cylinder 10 and can urge the second cylinder 16 distally away from the first cylinder 10 such that the inner cylinder 16 can be extended to the extended irreversible position. It will be appreciated that the first track 20 is shown by way of example only, where the first track 20 may have any suitable shape and configuration. It will be appreciated that one or a plurality of tracks may be incorporated to achieve a similar operation and it will be appreciated that the track and/or projection can be associated with any suitable component.

Example embodiments of the automatic non-reusable needle guard 8 can also include a locking mechanism (FIGS. 8-10) that can prevent retraction of the second cylinder 16, relative to the first cylinder 10, after the second cylinder 16 is fully extended in the extended irreversible position. A flexible hinge 24 can be associated with the outer portion 14 of the first cylinder 10 where the flexible hinge 24 can be biased radially inward. In the initial position, the flexible hinge 24 can be biased inwardly in its resting state (FIG. 8) in a first position. In the released or armed position (FIG. 9) the flexible hinge 24 can be flexed radially outwardly into a second position as the second cylinder 16 is rotated and axially translated relative to the first cylinder 10. In the extended irreversible position (FIG. 10), the flexible hinge 24 can flex radially inward into a third position such that the flexible hinge 24 can engage a slot 34 in the second cylinder 16. In one embodiment, when the flexible hinge 24 has flexed radially inward into the third position and has engaged the slot 34, the second cylinder 16 cannot be moved proximally to expose the distal tip of the needle due to the abutment between the flexible hinge 24 and the slot 34. Such a configuration may prevent accidental needle sticks after use of the needle guard 8.

The foregoing description of embodiments and examples has been presented for purposes of illustration and description. It is not intended to be exhaustive or limiting to the forms described. Numerous modifications are possible in light of the above teachings. Some of those modifications have been discussed, and others will be understood by those skilled in the art. The embodiments were chosen and described in order to best illustrate principles of various embodiments as are suited to particular uses contemplated. The scope is, of course, not limited to the examples set forth herein, but can be employed in any number of applications and equivalent devices by those of ordinary skill in the art. Rather it is hereby intended the scope of the invention to be defined by the claims appended hereto.

We claim:

1. A safety system comprising
(a) a needle retaining device having a proximal end, a distal end, and a needle fixedly coupled to the distal end of the needle retaining device;
(b) a needle guard assembly, the needle guard assembly being configured for attachment to the distal end of the needle retaining device, the needle guard assembly comprising;
(i) a first cylinder, wherein the first cylinder is attached to the distal end of the needle retaining device; and
(ii) a second cylinder, wherein the second cylinder is selectively movable relative to the first cylinder from a first position, to a second position wherein the needle is fully exposed, to a third position wherein the needle is fully covered.

2. The safety system of claim 1, wherein the needle is partially exposed in the first position.

3. The safety system of claim 1, wherein the needle is fully covered in the first position.

4. The safety system of claim 1, wherein the needle guard assembly is configured for attachment to the needle retaining device in a one step assembly.

5. The safety system of claim 1, wherein the first cylinder includes an inner portion that is configured to be coupled to the distal end of the needle retaining device and is positioned inside the second cylinder and an outer portion that is positioned outside the second cylinder.

6. The safety system of claim 1, wherein the second cylinder comprises at least one proximally extending flange, wherein the at least one proximally extending flange is configured to stabilize the needle guard assembly relative to the needle retaining device.

7. The safety system of claim 1, the first cylinder having a track and the second cylinder having a projection, wherein the projection engages the track such that the track guides the movement of the second cylinder relative to the first cylinder during operation of the needle guard assembly.

8. The safety system of claim 1, wherein the second cylinder is locked in the third position such that the second cylinder is prevented from moving proximally.

9. The safety system of claim 1, further comprising a flexible hinge, wherein the needle guard assembly is locked in the third position with the flexible hinge.

10. The safety system of claim 1, wherein the needle guard assembly comprises a track having a substantially J-shaped configuration.

11. The safety system of claim 1, further comprising a flexible hinge, wherein the flexible hinge is in a relaxed state in the first position.

12. The safety system of claim 1, further comprising a spring configured to bias the needle guard assembly in a generally distal direction.

13. The safety system of claim 1, wherein the second cylinder is configured to stabilize the needle guard assembly relative to the needle retaining device.

14. The safety system of claim 13, wherein the second cylinder at least partially covers the needle retaining device in the second position such that the needle guard assembly is substantially stabilized.

15. The safety system of claim 1, wherein the needle guard assembly comprises a track having a substantially linear configuration.

16. A method for assembling a safety system comprising:
providing a needle retaining device having a proximal end, a distal end, and a needle fixedly coupled to the distal end of the needle retaining device;
providing a needle guard assembly, the needle guard assembly being configured for attachment to the distal end of the needle retaining device, the needle guard assembly comprising a first cylinder, wherein the first cylinder is attached to the distal end of the needle retaining device, and a second cylinder, wherein the second cylinder is selectively movable relative to the first cylinder from a first position, to a second position wherein the needle is fully exposed, to a third position wherein the needle is fully covered; and
coupling the needle guard assembly and the needle retaining device.
